(19) United States
(12) Patent Application Publication
(10) Pub. No.: US 2024/0005428 A1
(43) Pub. Date: Jan. 4, 2024

(54) GENOMIC GRAVE AND FUNERAL SYSTEM FOR CREATION OF GENOMIC GRAVE

(71) Applicant: Yun Kyung LEE, Goyang-si, Gyeonggi-do (KR)

(72) Inventor: Yun Kyung LEE, Goyang-si, Gyeonggi-do (KR)

(21) Appl. No.: 18/254,534

(22) PCT Filed: Nov. 26, 2021
(86) PCT No.: PCT/KR2021/017616

§ 371 Date: Sep. 21, 2023

(30) Foreign Application Priority Data
Nov. 26, 2020 (KR) 10-2020-0161196
Nov. 26, 2021 (KR) 10-2021-0165209

(51) Int. Cl. G06Q 50/18

(52) U.S. Cl. CPC G06Q 50/186

(57) ABSTRACT

A funeral system using genomic information includes a genomic grave creation unit configured to request a grave manufacturer terminal to create a genomic grave made of a material including a genome of a deceased person and an artificial nucleic acid sequence, which is formed by converting authentication information into a nucleic acid sequence on the basis of a preset correspondence between an information unit and nucleic acid sequence information, in accordance with a request from a customer terminal, and a genomic information management unit configured to control access to information about the deceased person using authentication information corresponding to the artificial nucleic acid sequence.

### TECHNICAL FIELD

The present disclosure relates to a genomic grave using a genome and a system for creation of the genomic grave.

### BACKGROUND ART

Although science technology is being developed every day, humans unfortunately helplessly die due to natural misfortunes such as disasters, an earthquake, volcano eruption, and diseases, and artificial disasters caused by environmental destruction. Even though science technology gives priority to prevention, medical treatment, expectation, planning, etc., it can be seen that measures and technologies for coping with corpses, which greatly increase due to disasters, are not globally satisfactory from all countries that have taken measures against situations during the pandemic of COVID-19 in 2020.

Accordingly, there is a need for changing paradigms and creating a new funeral culture in preservation of a corpse and remembrance.

In relation to this fact, a bone crystal manufacture system for manufacturing a crystal using bones has been disclosed in Korean Patent Application No. 10-2010-0011568, the system including: a crusher that simultaneously performs a crushing process of crushing bones left after cremation and a drying process of removing moisture by instantaneously heating the bones, measures the size of particles of the crushed bones, and sends out only bone powder under 0.1 mm; a mixer that adds and mixes the bone powder sent from the crusher, a mixture such as a low corrosive material and an eco-friendly material, and an additive such as a coating agent; a former that includes a shaper receiving the bone powder mixed by the mixture and producing the bone powder into a crystal through press-forming and a surface finisher coating or plating the surface of the crystal to prevent corrosion; a controller that control whether to driving the crusher, the mixture, and the former such that bones are crushed, mixed, and formed on the basis of set data transmitted from a data input unit; and a display that shows the processes of crushing, mixing, and forming bones in cooperation with the controller.

Further, an apparatus for making a bead-shaped crystal from bones left after cremation has been disclosed in Korean Patent Application No. 10-2009-0039122, the apparatus including a heater 11 that is disposed at an upper portion in a body 10 and melts bones through heating and a molten material re-crystallizer 35 that is disposed at a lower portion in the body 10 and re-crystallizes a molten bone solution into a small bead size, in which the heater 11 has a case 12 having several magnetrons 13, a heating case 14 generating heat using microwaves generated by the magnetrons 13 in the case 12, and a melting pot 15 in the heating case 14, an inner insulation layer 18 outside the heating case 14, an outer insulation layer 19 between the inner insulation layer 18 and the case 12, and a bottom insulation layer 20 on the bottom of the case 12; the melting pot 15 has a pot cover 16, a gas exhaust pipe 21 is disposed at the center of the pot cover 16, a stirring shaft 22 having a stirrer 24 at an end is disposed in the gas exhaust pipe 21, and a driving motor 25 driving the stirring shaft 22 is disposed at the upper end of the stirring shaft 22; and the pot cover 16 has a supply pipe 27 and a hopper 28 for supplying bones into the melting pot 15, the supply pipe 27 has a feeding amount adjuster 29 adjusting the feeding amount of bones and a feeding amount activator 30 operating the feeding amount adjuster 29, a molten material outlet 15-1 is formed at the lower portion of the melting pot 15, an outlet cap 31 is disposed at the molten material outlet 15-1, and an outlet cap actuator 32 moving the outlet cap 31 up and down is provided.

### DISCLOSURE

### Technical Problem

However, according to the prior art documents, they all use the bones of a deceased person, so it is difficult to figure out a useful value except using them simply for memorials. Further, since a crystal is formed by processing the bones themselves of a deceased person, the processing method is very difficult. Further, it is difficult to leave a mark of a deceased person on a crystal itself made by processing bones, so it is required to leave a mark showing that it is a deceased person on the outside of a crystal, and in this case, there is a possibility of mark being damaged.

### Technical Solution

A genomic grave according to an embodiment of the present disclosure is a genomic grave made of a material including a genome of a deceased person and an artificial nucleic acid sequence and the artificial nucleic acid sequence is formed by converting authentication information into a nucleic acid sequence on the basis of a preset correspondence between an information unit and nucleic acid sequence information.

The genomic grave may be any one of a sculpture, a flowerpot, a picture, and a capsule.

The genomic grave may be configured such that a plurality of unit elements forms one work, and the unit elements may include a genome and an artificial nucleic acid sequence for a plurality of deceased people, respectively.

The genomic grave genomic grave may form one work through materials of a plurality of pictures, and the materials of a plurality of pictures may include a genome and an artificial nucleic acid sequence for a plurality of deceased people, respectively.

The genomic grave may be a picture including a plurality of sections and the sections may include genomes and artificial nucleic acid sequences for different deceased people, respectively.

The materials of pictures may be coloring materials or materials that are colored by the coloring materials.

The genomic grave may further include an artificial nucleic acid sequence formed by converting information about a deceased person into a nucleic acid sequence on the basis of a preset correspondence between an information unit and nucleic acid sequence information.

The information about a deceased person may include a will.

A funeral system using genomic information according to an embodiment of the present disclosure includes: a genomic grave creation unit configured to request a grave manufacturer terminal to create a genomic grave made of a material including a genome of a deceased person and an artificial nucleic acid sequence, which is formed by converting authentication information into a nucleic acid sequence on the basis of a preset correspondence between an information unit and nucleic acid sequence information, in accordance with a request from a customer terminal; and a genomic information management unit configured to control access to information about the deceased person using authentication information corresponding to the artificial nucleic acid sequence.

The information about the deceased person may include at least one of genomic information of the deceased person, disease information of the deceased person, physical characteristic information of the deceased person, private personal information of the deceased person, a will of the deceased person, and the authentication information.

The information about the deceased person may further include information about a family of the deceased person.

Information about a plurality of deceased people may be stored at a plurality of nodes constituting a blockchain network.

The funeral system may further include a corpse treatment management unit configured to provide a corpse video of a deceased person taken along a movement path in a funeral to the customer terminal in real time.

The funeral system may further include: a funeral article proxy purchase unit configured to provide a funeral article list to the customer terminal and configured to, when a specific funeral article is selected through the customer terminal, transmit a purchase request to a company terminal of the selected funeral article; and a Q&A service provider configured to, when a question related to a funeral procedure is input from the customer terminal, analyze the input question and provide an answer to the input question.

The funeral system may further include an avatar management unit configured to create an avatar of a deceased person in a virtual space and control changing of avatar information on the basis of authentication information corresponding to the artificial nucleic acid sequence.

The genomic grave may be any one of a sculpture, a flowerpot, and a picture.

The genomic grave may be configured such that a plurality of unit elements forms one work, and the unit elements may include a genome and an artificial nucleic acid sequence for a plurality of deceased people, respectively.

The genomic grave genomic grave may form one work through materials of a plurality of pictures, and the materials of a plurality of pictures may include a genome and an artificial nucleic acid sequence for a plurality of deceased people, respectively.

The materials of pictures may be coloring materials or materials that are colored by the coloring materials.

The genomic grave may further include an artificial nucleic acid sequence formed by converting information about a deceased person into a nucleic acid sequence on the basis of a preset correspondence between an information unit and nucleic acid sequence information.

The information about a deceased person may include a will.

The funeral system may further include a virtual grave provider configured to provide a shape corresponding to the genomic grave in a virtual space through the customer terminal.

### Advantageous Effects

According to an embodiment of the present disclosure, it is possible to solve an environment problem and cost problem due to burying a corpse or keeping bone powder.

Further, it is possible to solve legal problems that are generated due to difficulty in authentication of a corpse when a lot of time passes after burial or a funeral.

Further, it is possible to create a genomic grave at many memorial places.

Further, since a genomic grave is manufactured using a material having an artificial nucleic acid sequence corresponding to authentication information and genomic information is managed using the authentication information, it is possible to link and use a genome and genomic information.

Further, since an artificial nucleic acid sequence corresponding to authentication information is included in a grave material itself, it is possible to prevent authentication information from being damaged or forged or falsified.

Further, since a grave material having an actual object of a genome and an artificial nucleic acid sequence is used, it is possible to manufacture a genomic grave into various types such as a sculpture, a flowerpot, a picture, and a capsule, and it is possible to manufacture a grave that is eco-friendly and has an excellent aesthetic effect.

According to an embodiment of the present disclosure, since there is provided a general funeral system that not only can create a genomic grave, but can treat a corpse, manage a funeral, and provide an avatar management service, it is possible to provide mental stability and convenience to a bereaved family and provide economic benefits by using genomic information.

### DESCRIPTION OF DRAWINGS

FIGS. 1A, 1B, 1C and 1D are views showing examples of a genomic grave according to an embodiment of the present disclosure.

FIG. 2 is a configuration diagram of a funeral system using genomic information according to an embodiment of the present disclosure.

FIG. 3 is a flowchart showing an example of a genomic grave creation process according to an embodiment of the present disclosure.

FIG. 4 is a flowchart showing an example of a genomic information management process according to an embodiment of the present disclosure.

FIG. 5 is a flowchart showing an example of a corpse treatment process according to an embodiment of the present disclosure.

FIG. 6 is a diagram showing the configuration of a funeral management unit 240 according to an embodiment of the present disclosure.

FIG. 7A is a flowchart showing an example of a funeral article proxy purchase process and FIG. 7B is a flowchart showing an example of a Q&A service process.

FIG. 8 is a diagram showing the configuration of an avatar management unit 250 according to an embodiment of the present disclosure.

### MODE FOR INVENTION

It would be considered that the method or configuration of an embodiment described herein can be implemented for another method of configuration described herein.

When a term is used with “including” in claims and/or the specification, the term may mean “one”, but coincides with meanings “one or more”, “at least one”, and “one or more than one”.

In use of the term “or” in claims, even though the description of the present disclosure supports definition saying simply selectable expressions and “and/or”, the selectable expressions are used to mean “and/or” unless the selectable expressions clearly mean exclusive or simply selectable objects.

The meanings of “transmission”, “delivery”, and “reception in claims and/or the specifications include not only directly transmitting, delivering, and receiving, but transmitting, delivering, and receiving with another component therebetween.

Other objects, characteristics, and advantages of the present disclosure will be made clear from the following detailed description. However, since various changes and modifications within the spirit and scope of the present disclosure will be made clear to those skilled in the art through the following detailed description, it should be understood that the detailed description and detailed examples represent detailed embodiments of the present disclosure, but are provided only examples. Various exemplary embodiments of the present disclosure will be discussed in detail below in relation to the accompanying drawings showing the exemplary embodiments of the present disclosure. Although detailed examples will be discussed, but this is for only examples. Those skilled in the art would be able to recognize that other components and configurations can be used without departing from the spirit and scope of the present disclosure. The same reference numbers indicate the same components throughout the specification.

Hereafter, exemplary embodiments of the present disclosure are described in detail with reference to the accompanying drawings.

A genome described herein may be a genome or a living object or a dead object. A genome includes not only genomic DNA such as nuclear DNA, whole genome DNA, partial genome DNA, and DNA of an unborn baby circulating in maternal blood extracted from a tissue or blood, but genome DNA such as nuclear DNA, whole genome DNA, partial genome DNA, and DNA of an unborn baby limited to a specific cell group extracted from specific cells such as somatic cells, germ cells, tumor cells, infectious cells of microorganisms or other organisms. Further, the genome is a substance that is extracted by a technology, which was developed to study genome DNA such as cDNA and specifically treated mRNA, and can be sequenced, and the definition thereof is not limited to the current technology.

Further, in the specification, genomic information includes at least one of phenotypes of various measured data in addition to characteristic information, pathological information, medical information, genome sequence information, genome experimental analysis data information, data mining analysis data information of a genome and a character or a disease, data information extracted through comparison with an existing genome database, and any observable phenotype for studying a genome and a meaningful phenotype. Genome sequence information may include a cDNA sequence, an mRNA sequence, an expression profile, epigenetic data, protein data, whole genome data, methylation data, metabolomic data, microbiome data, human body sequence data, genotype data from PCR, genotype data from DNA microarrays, genotype data from total genome sequencing, genotype data from whole genome sequencing, genotype data from gene sequencing, karyotype data, preimplantation genetic testing data, non-invasive prenatal genetic testing data of an embryo bud or an unborn baby, etc. The ranges of these data are not limited and can be obtained by methods generally known in the art.

A genomic grave according to an embodiment of the present disclosure is a genomic grave made of a material including a genome of a deceased person and an artificial nucleic acid sequence and the artificial nucleic acid sequence is formed by converting authentication information into a nucleic acid sequence on the basis of a preset correspondence between an information unit and nucleic acid sequence information. A term “deceased person” is used for the convenience, but not only a genome of human, but genomes of animals may be included. Further, the genomic grave may be formed during existence or after death using a genome provided from a “deceased person” when he/she is alive or may be taken from the corpse after death of a “deceased person”.

Depending on embodiments, an artificial nucleic acid sequence formed by converting information about a deceased person into a nucleic acid sequence on the basis of a preset correspondence between an information unit and nucleic acid sequence information may be further included. In this case, the information about a deceased person may include at least one of genomic information of the deceased person, disease information of the deceased person, physical characteristic information of the deceased person, and private personal information of the deceased person. Depending on embodiments, the information about a deceased person may include a will of the deceased person.

Hereafter, a manner of setting an artificial nucleic acid sequence and a manner of performing authentication are described in detail.

DNA or RNA that is the most fundamental unit constituting information in accordance with the present disclosure is a substance in which the fundamental structure of a nucleic acid is connected by phosphodiester bond, which is the same as the general definition in genetic engineering. Nucleic acids are bonded in a chain type by phosphodiester bond, thereby constituting DNA and RNA. A total of five kinds of compounds can be bonded to a base bonding portion in a nucleic acid structure, and, depending on what compound is bonded, DNA is composed of four kinds of bases of Adenine (A), Thymine (T), Guanine (G), and Cytosine (C) and RNA is composed of Adenine (A), Uracil (U), Guanine (G), and Cytosine (C). DNA and RNA are discriminated in accordance with whether —H or —OH is bonded to C-2 of a ring structure. In a body, specific amino acids are represented by three combinations of four kinds of DNA- or RNA-type bases and protein is produced by a specific combination of such amino acids. For reference, two or more one-kind bases may exist in the three combinations. Further, such nucleic acid sequence is formed by phosphodiester bond that is the bonding method of bases existing in the natural world.

As a plurality of methods that performs setting by matching the sequence combinations and information unit, for example, three methods to be described below may be considered.

The first setting method is a method that sequentially sets a series of information units in accordance with a number of times of repetitive connection until a predetermined allowed number of times of one or, two or more bases (sequence units) and that sets a specific base combination as a marker showing a bond of sequence combinations.

TABLE 1

| Sequence | Information |
|---|---|
| combination | unit |
| A | 1 |
| AA | 2 |
| AAA | 3 |
| C | 4 |
| CC | 5 |
| CCC | 6 |
| T | 7 |
| TT | 8 |
| TTT | 9 |
| G | 0 |
| ATG | Bonding |
| | marker |

[sequence units are A, T, C, G]

Table 1 shows an embodiment of a manner of setting a specific information unit in an artificial combination manner of bases and a method of representing value information using the setting manner. As shown in Table 1, the used bases are A, C, T, and G, a predetermined allowed number of times was set as 3, and ATG was used as a bonding marker. The marker ATG provides directionality in sequence analysis and may be used as an index that can show whether the direction of sequence analysis is correct, and for example, when an analysis matter includes a sequence combination of GTA, which means that analysis should be reversely performed. As shown in Table 1, when representing value information of ‘1480632’ that is a portion of a resident registration number, ‘A’, ‘C’, ‘TT’, etc. were used as corresponding sequence combinations to represent information units ‘1, ‘4’, ‘8’, etc., and the bonding marker ‘ATG’ was used between the sequence combinations to check the bonding types, etc. of the sequence combinations. Accordingly, by setting in advance a series of information units in certain combinations of sequence units, respectively, and configuring nucleic acid sequences of specific information data ‘1480632’ to represent the data, it is possible, as below, to easily check the value information.

Value information: 1480632

Nucleic acid sequence: A ATG C ATG TT ATG G ATG CCC ATG AAA ATG AA

A second setting manner, as in Table 2 and Table 3, is a manner that uses a specific base (sequence unit) as a start and/or end marker of a sequence combination and that sets sequential information units in accordance with one, or two or more numbers of times of repetitive connection or a combination of the other bases.

Table 2 shows another embodiment of a manner of setting a specific information unit in an artificial combination manner of bases and a method of representing value information using the setting manner.

TABLE 2

| Sequence | Information |
|---|---|
| combination | unit |
| AT | 1 |
| AAT | 2 |
| AAAT | 3 |
| AAAAT | 4 |
| AAAAAT | 5 |
| CT | 6 |
| CCT | 7 |
| CCCT | 8 |
| CCCCT | 9 |
| CCCCCT | 0 |
| T | Bonding |
| | marker |

[sequence units are A, C, and T]

As shown in Table 2, T was set as an end marker of sequence combinations, A and C were set as repetitive sequence units of sequence combinations, and a predetermined allowed number of times was set as 5. The information data of “1480632” that is the later term of the resident registration number are encoded in the manner of Table 2 as follows.

Value information: 1480632

Nucleic sequence: AT AAAAT CCCT CCCCCT CT AAAT AAT

Further, as shown in Table 3, A was set as a start marker of sequence combinations and two combinations of A, T, C, and G were set as sequence combinations. The information data of “1480632” are encoded in the manner of Table 3 as follows.

TABLE 3

| Sequence | Information |
|---|---|
| combination | unit |
| ACC | 1 |
| ACT | 2 |
| ACG | 3 |
| ATC | 4 |
| ATT | 5 |
| ATG | 6 |
| AGC | 7 |
| AGT | 8 |
| AGG | 9 |
| AAA | 0 |
| AT | Start |
| | marker |

[sequence units are A, C, T, and G]

Value information: 1480632

Nucleic acid sequence: ACC ATC AGT AAA ATG ACG ACT

The third setting manner, as shown in Table 4, is a manner of setting a specific information unit in two, or three or more sequence combinations of specific bases and of setting value information in the specific combinations.

Table 4 shows another embodiment of a manner of setting a specific information unit in an artificial combination manner of bases and a method of representing information data using the setting manner. As shown in Table 4, three bases are combined in the order of first base, second base, and third base, thereby representing one information unit (alphabet). Information units were expressed as A-Z in Table 4. For example, a sequence combination ‘ATT’ was made by a combination of a first base ‘A’, a second base ‘T’, and a third base ‘T’, and an information unit ‘K’ was assigned to the sequence combination. A sequence combination ‘CCC’ was set as an end marker to check whether a nucleic acid sequence has perfect information. Meanwhile, in order to represent perfection of a nucleic acid sequence, a sequence combination ‘CCC’ may be used as a start marker or, depending on cases, ‘CCC’ may be used as both start and end markers. When information data is “JONG IL LEE”, the information data are encoded as follows.

TABLE 4

| First | Second base | | | Third |
|---|---|---|---|---|
| base | A | T | C | base |
| A | A | J | S | A |
| | B | K | T | T |
| | C | L | U | C |
| T | D | M | V | A |
| | E | N | W | T |
| | F | O | X | C |
| C | G | P | Y | A |
| | H | Q | Z | T |
| | I | R | End | C |
| | | | marker | |

[sequence units are A, C, T, and G]

Value information: JONGILLEE

Nucleic acid sequence: ATA TTC TTT CAA CAC ATC ATC TAT TAT CCC

An artificial nucleic acid sequence of the present disclosure is obtained by selecting any one of the matching manners of sequence combinations and information units described above. However, the manners described above are only a portion of a great number of manners of matching sequence combinations and information units.

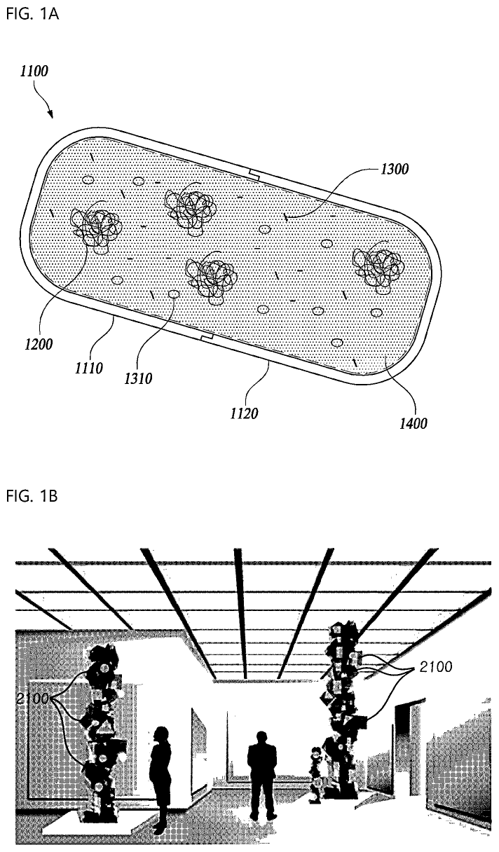

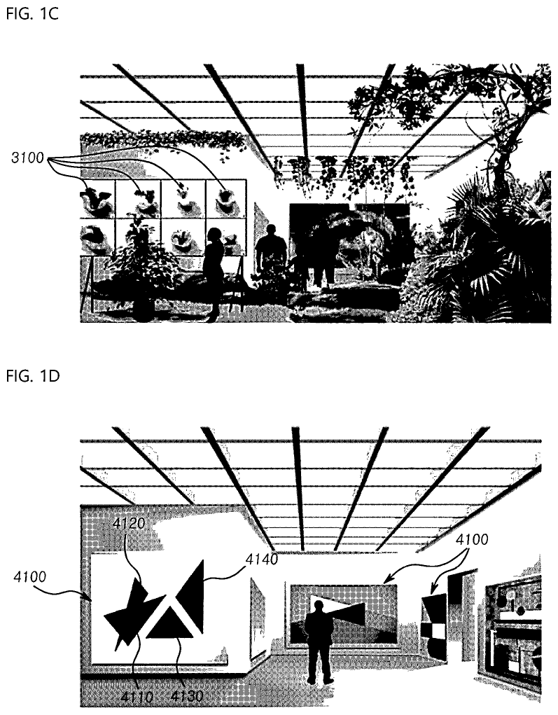

FIGS. 1A, 1B, 1C and 1D are views showing examples of a genomic grave according to an embodiment of the present disclosure. As shown in FIGS. 1A, 1B, 1C and 1D, a genomic grave may have at least one type of a sculpture, a flowerpot, a picture, and a capsule.

Referring to FIG. 1A, a genomic grave may have a capsule type.

A capsule 1100 is composed of a left capsule 1110 and a right capsule 1120 that are made of a material, which is not deformed by heat and external mechanical pressure, and can be separated from and combined with each other. Since many genome DNAs 1200 and artificial nucleic acid sequences 1300 of a specific deceased person distributed in a solution 1400 are included in the capsule 1100, even though some of the genome DNAs 1200 or artificial nucleic acid sequences 1300 are damaged, it is possible to perform authentication by analyzing the other many DNAs.

Meanwhile, as shown in FIG. 1A, artificial nucleic acid sequences may be provided in the capsule 1100 in a state 1310 in which they are bonded to vectors. The artificial nucleic acid sequences 1300 are composed of a much less number of bases than the genome DNAs 1200, so it is possible to simply analyze artificial nucleic acid sequences 1300 within a short time.

The vectors include all biological and physicochemical vectors to which amplification techniques other than PCR, which is a general technique that can amplify genome DNAs, can be applied, including various nucleic acid sequence linkage nano-particle vectors.

Referring to FIG. 1B, a genomic grave may have a sculpture type. The sculpture may have the shapes of a hexahedron, a pyramid, a sphere, etc.

A genomic grave may include a plurality of sculptures 2100 that form one work, and the sculptures 2100 may include a genome and an artificial nucleic acid sequence for a plurality of deceased people, respectively. That is, it can be seen that different sculptures 2100 may be graves of different deceased people.

These genomic graves may be used as memorials for memorizing an event causing many deceased people. For example, by figuring a type that can function as a mass grave and entirely makes it possible to memorize an event in order to memory The Korean War, the May 18 Gwangju Democratization Movement, Battle of Yeonpyeong, Cheonan Ship Attach, Sewol Ferry Disaster, etc., it is possible to have a function as a memorial too.

Although a work of art for exhibition was shown as a sculpture in FIG. 1B, various articles having a 3D shape such as an accessory may be used.

Referring to FIG. 1C, a genomic grave may be a flowerpot 3100.

A bereaved family can cherish the memory of a deceased person by implanting a plant in a flowerpot, which is made of a material including a genome of the deceased person and an artificial nucleic acid sequence, and seeing the plant growing in the flowerpot. The bereaved family can be healed by growth of the plant.

Referring to FIG. 1D, a genomic grave may be a picture 4100.

A bereaved family or a person who is requested by a bereaved family can manufacture the picture 4100 using a material of a picture including a genome of a deceased person and an artificial nucleic acid sequence. The material of a picture may be a coloring material such as a dye, a pigment, and a paint or a material that is colored by coloring materials such as paper, fabric, and canvas. Depending on embodiments, a genomic grave may be one work of art composed of a plurality of picture materials and the picture materials may include a genome and an artificial nucleic acid sequence for a plurality of deceased people, respectively.

As shown in FIG. 1D, a plurality of sections 4110, 4120, 4130, and 4140 may be included in one picture and each of the sections may include a genome and an artificial nucleic acid sequence for a specific deceased person. The sections in a picture may be expressed as figures such as a triangle, a rectangle, and a circle.

For example, the left picture in FIG. 1D may be a genomic grave for four deceased people. Paints corresponding to the FIGS. 4110, 4120, 4130, and 4140, respectively, may include genomes and artificial nucleic acid sequences for different deceased people, respectively. Alternatively, a red FIG. 4110 and black FIGS. 4120, 4130, and 4140 may include genomes and artificial nucleic acid sequences for different deceased people. That is, the black FIGS. 4120, 4130, and 4140 may include a genome and an artificial nucleic acid sequence for specific one deceased person and the red figure may include a genome and an artificial nucleic acid sequence for another deceased person.

Such a picture type genomic grave may be used as a family grave. The picture type genomic grave may have an eco-friendly aesthetic effect and represent features of only a family.

The types of genomic graves described above are only examples and it would be apparent to those skilled in the art that a genomic grave can be modified into various types. The sculpture, picture, flowerpot, and capsule described above are only exemplary types and a genomic grave may be manufactured in various types. For example, a manner that stabilizes a genome and authentication information in print ink through nano-particle connection or coating, prints the genome and authentication information, and reads the genome and authentication information using a fluorescent substance or a nano-reactant without sequence analysis may be used. Accordingly, a genomic grave may be printed on paper, etc. and kept in a book type or may be kept in a container like a seed storage such as a seed vault.

A genome and an artificial nucleic acid sequence constituting a genomic grave may be distributed throughout the material forming the genomic grave or may be included only in a portion of the genomic grave. For example, as described above, a genome and an artificial nucleic acid sequence may be included only in a partial section of a picture (a partial color or a portion of canvas) or a genome and an artificial nucleic acid sequence may be included only in a partial section of a sculpture. Further, when a sculpture is composed of a plurality of unit sculptures, a genome and an artificial nucleic acid sequence may be included only in some unit sculptures.

A genomic grave may be any one type of a sculpture, a picture, a flowerpot, and a capsule or may be an overlapping type of two or more thereof. For example, it may be possible to create a capsule type genomic grave and then mount the genomic grave on a sculpture, a picture, a flowerpot, or the like.

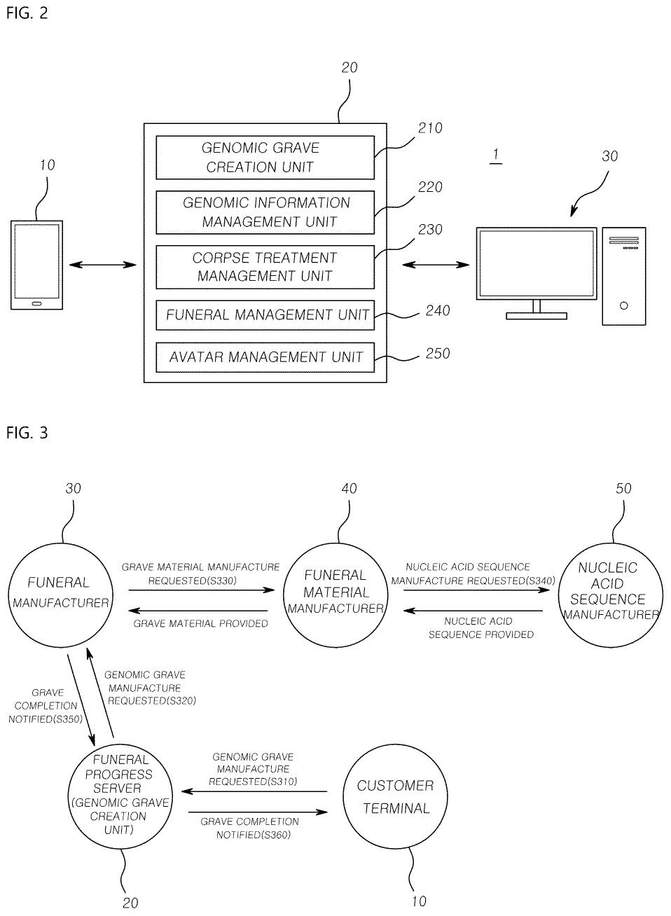

FIG. 2 is a configuration diagram of a funeral system using genomic information according to an embodiment of the present disclosure.

Referring to FIG. 2, a funeral system 1 includes a customer terminal 10, a funeral progress server 20, and a grave manufacturer terminal 30.

The customer terminal 10 is a device that can communicate with the funeral progress server 20 through wired or wireless communication, and for example, may be a mobile phone, a computer, a laptop, etc. The customer terminal 10 can request the funeral progress server 20 to create a genomic grave. Further, the customer terminal 10 can request information about a deceased person from the funeral progress server 20 while transmitting authentication information. Further, the customer terminal 10 can be provided with a corpse video of a deceased person taken along a movement path in a funeral. Further, the customer terminal 10 can connect with the funeral progress server 20 and can purchase funeral articles from a funeral article list or can be provided with a funeral service such as questioning and receiving answers. Further, the customer terminal 10 can change avatar information of a deceased person created by the funeral progress server 20 using authentication information.

All of the functions of the customer terminal 10 may be performed in one customer terminal. Alternatively, only one customer terminal 10 is shown in FIG. 2, but only some of the functions described above may be performed in a plurality of customer terminals 10. For example, the customer terminal 10 that requests creation of a genomic grave, receives a corpse video, and is provided with a funeral service may be a terminal of a bereaved family and the customer terminal 10 that requests information about a deceased person may be a terminal of a genomic information management company.

The funeral progress server 20 includes: a genomic grave creation unit that requests the grave manufacturer terminal 30 to create a genomic grave made of a material including a genome of a deceased person and an artificial nucleic acid sequence, which is formed by converting authentication information into a nucleic acid sequence on the basis of a preset correspondence between an information unit and nucleic acid sequence information, in accordance with a request from the customer terminal 10; and a genomic information management unit 220 that controls access to information about a deceased person using authentication information corresponding to the artificial nucleic acid sequence. In addition, the funeral progress server 20 may include: a corpse treatment management unit 230 that provides a corpse video of a deceased person taken along a movement path in a funeral to the customer terminal in real time; a funeral management unit 240 that enables a customer to purchase funeral articles by providing a funeral article list to the customer terminal 10 or provides a funeral service such as providing answers to questions input through the customer terminal 10; and an avatar management unit 250 that creates an avatar of a deceased person in a virtual space and controls changing of the avatar information on the basis of authentication information corresponding to the artificial nucleic acid sequence.

The funeral progress server 20 may include a processor, a memory, a communication unit for communicating with the customer terminal 10 and the grave manufacturer terminal 30, and an input/output unit for receiving input from a user or outputting an image or a sound. The processor performs the functions of the funeral progress server 20 described above and the memory performs a function of storing data when the functions described above are performed.

The grave manufacturer terminal 30 is a device that can communicate with the funeral progress server 20 through wired or wireless communication, and for example, may be a mobile phone, a computer, a laptop, etc. The grave manufacturer terminal 30 receives a grave manufacture request from the funeral progress server 20, and when a grave is completed, the grave manufacturer terminal 30 notifies the funeral progress server 20 of the fact. Only one grave manufacturer terminal 30 is shown in FIG. 2, but a plurality of grave manufacturer terminals may be included, depending on the kinds of genomic graves.

Though not shown in FIG. 2, a grave material manufacturer and a nucleic acid sequence manufacturer may be additionally included. This is described in detail in relation to FIG. 3.

FIG. 3 is a flowchart showing an example of a genomic grave creation process according to an embodiment of the present disclosure.

Referring to FIG. 3, a genomic grave manufacture request is received the genomic grave creation unit 210 from a customer terminal 10 (S310). The genomic grave creation unit 210 can receive a genomic grave manufacture request through the communication unit of the funeral progress server 20. When a genomic grave manufacture is requested, authentication information may be provided from the customer terminal 10.

The genomic grave creation unit 210 requests the grave manufacturer terminal 30 to manufacture a genomic grave (S320).

The grave manufacturer terminal 30 requests a grave material manufacturer terminal 40 to manufacture a grave material including a genome and an artificial nucleic acid sequence corresponding to the authentication information in accordance with a genomic grave manufacture request from the genomic grave creation unit 210 (S330).

The grave material manufacturer terminal 40 requests a nucleic acid sequence manufacturer terminal 50 to manufacture an artificial nucleic acid sequence corresponding to the authentication information provided from a customer (S340).

Accordingly, the nucleic acid sequence manufacturer manufactures and provides an artificial nucleic acid sequence corresponding to the authentication information to the grave material manufacturer 40. The grave material manufacturer 40 manufactures a grave material including the actual object of the genome provided from the customer and the artificial nucleic acid sequence provided from the nucleic acid sequence manufacturer 50, and provides the manufactured grave material to the grave manufacturer 30.

The grave manufacturer manufactures a genomic grave using the grave material provided from the grave material manufacturer and notifies the genomic grave creation unit 210 that a genomic grave has been completed (S350).

The genomic grave creation unit 210 notifies the customer terminal 10 that a genomic grave has been completed (S360).

It was described in this embodiment that a grave manufacturer, a grave material manufacturer, and a nucleic acid sequence manufacturer are different subjects, but at least two or more of manufacturing of a grave, manufacturing of a grave material, and manufacturing of an artificial nucleic acid sequence may be performed by the same subject. For example, manufacturing of a grave, manufacturing of a grave material, and manufacturing of an artificial nucleic acid sequence may be performed by the same subject. In this case, step S330 and step S340 may be omitted. Alternatively, manufacturing of a grave and manufacturing of a grave material may be performed by the same subject, and manufacturing of an artificial nucleic acid sequence may be performed by another subject. In this case, step S330 may be omitted. Alternatively, manufacturing of a grave material and manufacturing of an artificial nucleic acid sequence may be performed by the same subject, and manufacturing of a grave may be performed by another subject. In this case, step S340 may be omitted.

The case in which only an artificial nucleic acid sequence corresponding to authentication information is included in a genomic grave was described in this embodiment, but a genomic grave may further include an artificial nucleic acid sequence corresponding to information about a deceased person. In this case, when an artificial nucleic acid sequence corresponding to authentication information is manufactured, an artificial nucleic acid sequence corresponding to information about a deceased person can also be manufactured and provided. In this case, the information about a deceased person may include a will.

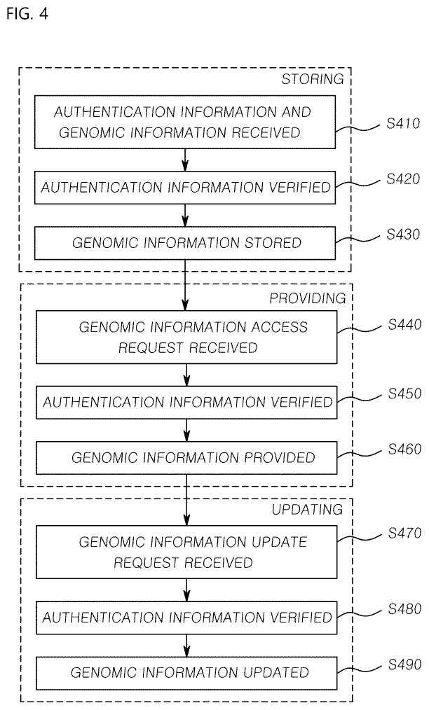

FIG. 4 is a flowchart showing an example of a genomic information management process according to an embodiment of the present disclosure.

First, a process of storing genomic information is described.

The genomic information management unit 220 receives authentication information and genomic information from a customer terminal 10 (S410). The genomic information management unit 220 can receive authentication information, and genomic information as information about a deceased person through the communication unit of the funeral progress server 20. The authentication information may be provided from the customer terminal 10 together with a genomic grave manufacture request.

The genomic information management unit 220 verifies the authentication information (S420) and then stores the genomic information (S430). In this case, the genomic information may be stored in a database connected inside or outside the funeral progress server 20 or may be stored in a blockchain network. Depending on embodiments, information about a plurality of deceased person may be stored at a plurality of nodes constituting a blockchain network. For example, information about a deceased person may be stored to correspond to nodes one to one. Alternatively, information about one or more deceased people may be stored at a specific node. In this case, the information about deceased person may include at least one of genomic information of the deceased person, disease information of the deceased person, physical characteristic information of the deceased person, and private personal information of the deceased person. Depending on embodiments, the information about a deceased person may include a will of the deceased person.

Next, a process of providing genomic information is described.

A genomic information access request is received at the genomic information management unit 220 from a customer terminal 10 (S440). In this case, authentication information is also received from the customer terminal 10. The customer terminal 10 in this case may be the same as or different from the customer terminal 10 that has transmitted genomic information. For example, when a bereaved family stores genomic information and then checks the stored information, the customer terminal 10 that has transmitted genomic information and the customer terminal that has made a genomic information access request would be the same. When a bereaved family stores genomic information and then provides authentication information to a genomic information management company and the genomic information management company makes a genomic information access request, the customer terminal 10 that has transmitted genomic information and the customer terminal 10 that has made a genomic information access request would be different.

The genomic information management unit 220 verifies the authentication information (S450) and then provides the genomic information to the customer terminal 10 (S460).

Next, a process of updating genomic information is described.

A genomic information update request is received at the genomic information management unit 220 from a customer terminal 10 (S470). In this case, authentication information is also received from the customer terminal 10. The customer terminal 10 in this case may be the same as or different from the customer terminal 10 that has transmitted genomic information. For example, a bereaved family can create a genomic grave and then additionally store information such as a will. Alternatively, a bereaved family may create a genomic grave and then a genomic information management company may create new information on the basis of the result of analyzing previously stored genomic information and then additionally store the new information.

The genomic information management unit 220 verifies the authentication information (S480) and then updates the genomic information (S490).

The processes of storing genomic information (S410˜S430), providing genomic information (S440˜S460), and updating genomic information (S470˜S490) were sequentially described with reference to FIG. 4, but the process of updating genomic information (S470˜S490) is not necessarily performed after the providing of genomic information (S440˜S460) and has only to be performed after the storing of genomic information (S410˜S430). Further, the providing of genomic information (S440˜S460) may be performed after the updating of genomic information (S470˜S490).

In this embodiment, representing “genomic information” may include not only information related to a genome of a deceased person, but various items of information related to the deceased person such as disease information of the deceased person, physical information of the deceased person, a will of the deceased person, and private personal information of the deceased person. Further, the genomic information may include not only information of a deceased person himself/herself, but information about the family of the deceased person. Further, the genomic information may include also authentication information corresponding to an artificial nucleic acid sequence.

The information about the family of a deceased person may include at least one of genomic information of the family of the deceased person, disease information of the family of the deceased person, physical characteristic information of the family of the deceased person, and private personal information of the family of the deceased person. Depending on embodiments, the information about the family of a deceased person may include a will of the family of the deceased person.

The information about a deceased person may be stored in an actual figure type of a nucleic acid sequence in a genomic grave and may be stored in a digital type in the genomic information management unit 220.

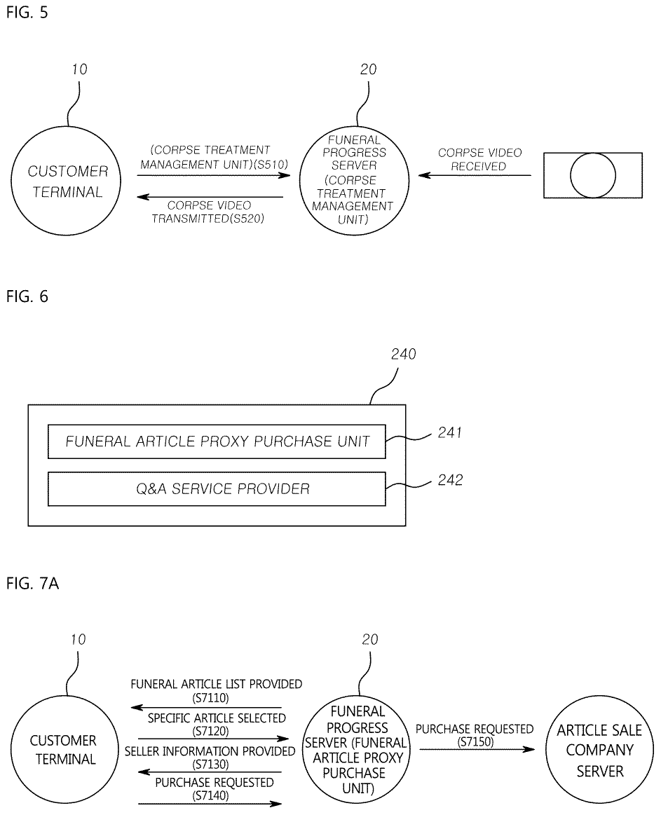

FIG. 5 is a flowchart showing an example of a corpse treatment process according to an embodiment of the present disclosure.

Referring to FIG. 5, a corpse treatment video request is received at the corpse treatment management unit 230 from a customer terminal 10 (S510). The corpse treatment management unit 230 can receive a corpse treatment video request through the communication unit of the funeral progress server 20.

The corpse treatment management unit 230 receives a corpse video of a deceased person taken along a movement path in a funeral and transmits the corpse video to the customer terminal 10 (S520). Cameras such as a CCTV are installed along a movement path including a funeral parlor, the inside of a car carrying a casket, and a crematorium, so a corpse video can be continuously taken. A bereaved family can feel easy by checking the process of moving and treating the corpse through the terminal 10. The corpse treatment management unit 230 can receive a corpse video regardless of a request from the customer terminal 10 and can provide the corpse video in accordance with a request from the customer terminal 10. Alternatively, the corpse treatment management unit 230 may receive a corpse video in accordance with a request from the customer terminal 10 and then provide the corpse video in accordance with a request from the customer terminal 10.

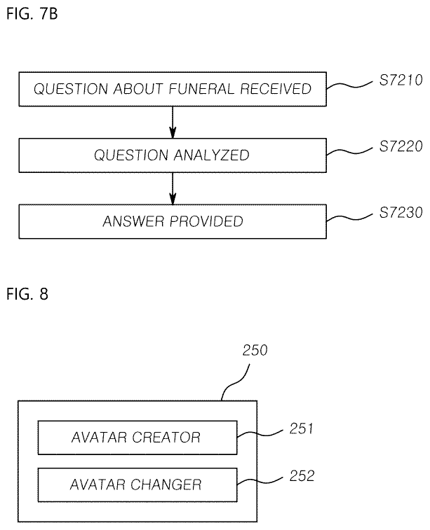

FIG. 6 is a diagram showing the configuration of the funeral management unit 240 according to an embodiment of the present disclosure, FIG. 7A is a flowchart showing an example of a funeral article proxy purchase process, and FIG. 7B is a flowchart showing an example of Q&A service process.

Referring to FIG. 6, the funeral management unit 240 may include at least one of: a funeral article proxy purchase unit 241 that provides a funeral article list to a customer terminal, and, when a specific funeral article is selected through the customer terminal, transmits a purchase request to a company terminal of the funeral article; and a Q&A service provider 242 that, when a question related to a funeral procedure is input from the customer terminal, analyzes the input question and provides an answer to the input question.

Referring to FIG. 7A, the funeral article proxy purchase unit 241 provides a funeral article list to a customer terminal 10 (S7110). Then, when a specific article is selected through the customer terminal 10 (S7120), the funeral article proxy purchase unit 241 provides the information of one or more sellers that sell the selected article to the customer terminal 10 (S7130). A bereaved family selects one appropriate company of the sellers and makes a purchase request for the selected article to through the customer terminal 10 (S7140). Accordingly, the funeral article proxy purchase unit 241 makes a purchase request online to the article sale company (S7150). Making a purchase request to an article sale company from the customer terminal 10 through the funeral article proxy purchase unit 241 was shown in FIG. 7A, but it may be possible to make a purchase request directly to an article sale company from the customer terminal 10. According to this embodiment, since a funeral article

list is provided by the funeral article proxy purchase unit 241, a bereave family can get all of articles for a funeral procedure. Further, since various choices for specific articles are provided, it is possible to purchase desired articles at appropriate prices.

Referring to FIG. 7B, when a question about a funeral procedure is input from a customer terminal 10 (S7210), the Q&A service provider 242 analyzes the input question (S7220) and provides an answer to the input question (S7230). The Q&A service provider 242 may provide such a Q&A

service by making Chatbot be executed in the customer terminal 10. Alternatively, the Q&A service may be provided through a message service such as a Short Messaging Service (SMS) or a Multimedia Messaging Service (MMS), or a Social Network Service (SNS). The Q&A service provider 242 may construe questions input through the customer terminal 10 using natural language analysis or may provide a plurality of questions to the customer terminal 10 to make the customer select specific questions.

FIG. 8 is a diagram showing the configuration of the avatar management unit 250 according to an embodiment of the present disclosure.

Referring to FIG. 8, the avatar management unit 250 may include an avatar creator 251 and an avatar changer 252.

The avatar creator 251 enables a customer terminal 10 to create an avatar of a deceased person in a virtual space. For example, the avatar creator can provide image candidates that can be used as an avatar of a deceased person and can create an avatar of the deceased person using an image selected from the provided image candidates by the customer terminal 10. Alternatively, the avatar creator 251 may create an avatar of a deceased person using an image provided from the customer terminal 10.

The avatar changer 252 change the information of an avatar in accordance with information provided from the customer terminal 10. For example, it is possible to change portions—a skin color, a hair color, clothing, or the like—of a created avatar. Alternatively, it may be possible to replace an avatar itself with another avatar.

The avatar creator 251 or the avatar changer 252 may be based on authentication information when creating an avatar or changing an avatar.

Alternatively, the funeral progress server 20 may further include a virtual grave provider (not shown) that provides a shape corresponding to a genomic grave in a virtual space through the customer terminal 10.

The virtual grave provider can provide a grave of a shape that a customer wants and that corresponds to the actual object of a genomic grave. A virtual grave may have various types such as the sculpture, picture, flowerpot, and capsule described above. The capsule described above means only an actual capsule and a physicochemical capsule, but, according to this embodiment, a virtual capsule having digital information (information about a deceased person) may be provided as a genomic grave.

A virtual grave takes little efforts and costs for maintaining and keeping and a customer can access the grave anytime in a virtual space. Consequently, only a virtual genomic grave may be provided without an actual object of a genomic grave.

Although the present disclosure was described in detail through embodiments, the present disclosure is not limited thereto and it is apparent to those skilled in the art that the present disclosure may be changed and applied in various ways without departing from the spirit of the present disclosure. Accordingly, the protective range of the present disclosure should be construed by the following claims and the scope and spirit of the present disclosure should be construed as being included in the patent right of the present disclosure.

1. A genomic grave made of a material including a genome of a deceased person and an artificial nucleic acid sequence,
wherein the artificial nucleic acid sequence is formed by converting authentication information into a nucleic acid sequence on the basis of a preset correspondence of an information unit and nucleic acid sequence information.

2. The genomic grave of claim 1, wherein the genomic grave includes at least one of a sculpture, a flowerpot, a picture, and a capsule.

3. The genomic grave of claim 1, wherein the genomic grave is configured such that a plurality of unit elements forms one work, and
the unit elements include a genome and an artificial nucleic acid sequence for a plurality of deceased people, respectively.

4. The genomic grave of claim 1, wherein the genomic grave forms one work through materials of a plurality of pictures, and
the materials of a plurality of pictures include a genome and an artificial nucleic acid sequence for a plurality of deceased people, respectively.

5. The genomic grave of claim 1, wherein the genomic grave is a picture including a plurality of sections and the sections include genomes and artificial nucleic acid sequences for different deceased people, respectively.

6. The genomic grave of claim 4 or 5, wherein the materials of pictures are coloring materials or materials that are colored by the coloring materials.

7. The genomic grave of claim 1, further comprising an artificial nucleic acid sequence formed by converting information about a deceased person into a nucleic acid sequence on the basis of a preset correspondence between an information unit and nucleic acid sequence information.

8. The genomic grave of claim 7, wherein the information about a deceased person includes a will.

9. The genomic grave of claim 7, wherein the information about a deceased person includes information of a bereaved family of the deceased person.

10. A funeral system using genomic information comprising:
a genomic grave creation unit configured to request a grave manufacturer terminal to create a genomic grave made of a material including a genome of a deceased person and an artificial nucleic acid sequence, which is formed by converting authentication information into a nucleic acid sequence on the basis of a preset correspondence between an information unit and nucleic acid sequence information, in accordance with a request from a customer terminal; and
a genomic information management unit configured to control access to information about the deceased person using authentication information corresponding to the artificial nucleic acid sequence.

11. The funeral system of claim 10, wherein the information about the deceased person includes at least one of genomic information of the deceased person, disease information of the deceased person, physical characteristic information of the deceased person, private personal information of the deceased person, personal authentication key information, a will of the deceased person, and the authentication information.

12. The funeral system of claim 11, wherein the information about the deceased person further includes information about a family of the deceased person.

13. The funeral system of claim 10, wherein at least one of information about a plurality of deceased people and information about families is stored at a plurality of nodes constituting a blockchain network.

14. The funeral system of claim 10, further comprising a corpse treatment management unit configured to provide information and a video about a corpse of a deceased person taken along a movement path in a funeral to the customer terminal in real time.

15. The funeral system of claim 10, further comprising at least one of:
a funeral article proxy purchase unit configured to provide a funeral article list to the customer terminal and configured to, when a specific funeral article is selected through the customer terminal, transmit a purchase request to a company terminal of the selected funeral article; and
a Q&A service provider configured to, when a question related to a funeral procedure is input from the customer terminal, analyze the input question and provide an answer to the input question.

16. The funeral system of claim 10, further comprising an avatar management unit configured to create an avatar of a deceased person in a virtual space and control changing of avatar information on the basis of authentication information corresponding to the artificial nucleic acid sequence.

17. The funeral system of claim 10, wherein the genomic grave is any one of a sculpture, a flowerpot, and a picture.

18. The funeral system of claim 10, wherein the genomic grave is configured such that a plurality of unit elements forms one work, and
the unit elements include a genome and an artificial nucleic acid sequence for a plurality of deceased people, respectively.

19. The funeral system of claim 10, wherein the genomic grave forms one work through a plurality of picture materials, and
the materials of pictures include a genome and an artificial nucleic acid sequence for a plurality of deceased people, respectively.

20. The funeral system of claim 19, wherein the materials of pictures are coloring materials or materials that are colored by the coloring materials.

21. The funeral system of claim 10, wherein the genomic grave further includes an artificial nucleic acid sequence formed by converting information about a deceased person on the basis of a preset correspondence between an information unit and nucleic acid sequence information.

22. The funeral system of claim 21, wherein the information about a deceased person includes a will.

23. The funeral system of claim 21, wherein the information about a deceased person includes information of a bereaved family of the deceased person.

24. The funeral system of claim 10, further comprising a virtual grave provider configured to provide a shape corresponding to the genomic grave in a virtual space through the customer terminal.
